(19) United States
(12) Patent Application Publication
(10) Pub. No.: US 2008/0292613 A1
(43) Pub. Date: Nov. 27, 2008

(54) Food Supplement Based on Group B Vitamins, Trace Elements and -3 and/or 6- Fatty Acids and Use of This Food Supplement in the Simultaneous Treatment of Localized Cellulite and Slackening of the Skin

(75) Inventors: Philippe Msika, Versailles (FR); Nathalie Piccardi, Arceau (FR); Pascale Cochet, Carrieres Sur Seine (FR); Michel Moreau, Orphin (FR)

Correspondence Address: FOLEY AND LARDNER LLP;SUITE 500, 3000 K STREET NW, WASHINGTON, DC, 20007, US

(73) Assignee: LABORATOIRES EXPANSCIENCE

(21) Appl. No.: 12/097,505

(22) PCT Filed: Dec. 15, 2006
(86) PCT No.: PCT/EP06/69770

§ 371 Date: Jun. 13, 2008

(30) Foreign Application Priority Data
Dec. 16, 2005 (FR) 0512799

(51) Int. Cl. A61K 38/48; A61K 33/30; A61K 33/06; A61K 33/34; A61K 33/04; A61P 3/04; A61K 33/24; A61K 33/26; A61K 33/32

(52) U.S. Cl. 424/94.65; 424/641; 424/682; 424/630; 424/702; 424/655; 424/646; 424/639

(57) ABSTRACT

The present invention relates to the use of a combination of group B vitamins, trace element(s), at least one oil comprising essential Ω-3 and/or Ω-6 fatty acids and an excipient suitable for oral administration, for use in the simultaneous treatment of excess weight and slackening of the skin localized in the same area. The invention also relates to a corresponding food supplement.

The invention relates to the use of a mixture of trace elements and group B vitamins, administered orally, for the simultaneous treatment of localised cellulite and slackening of the skin. The invention also relates to a cosmetic treatment method, administered orally, for excess weight localised in a surface area and simultaneously of slackening of the skin localised in the same area. The invention also relates to a food supplement, which may be administered within the scope of said use or said cosmetic treatment method.

Localised excess weight is materialised in the form of fat, the quantity and distribution whereof differ according to gender. In this way, adipose tissue represents 20 to 30% of body weight in women and 10 to 15% in men. Subcutaneous fat is twice as thick in women as in men. In men, fat accumulates around and above the waist (android distribution, metabolic risk factor) and below the waist, in the gluteofemoral region in women (gynoid distribution, not correlated with a vascular risk). One of the characteristics is this fat accumulated in the lower body is that it is difficult to mobilise. It is intended to provide reproductive energy needs (pregnancy and, above all, breastfeeding) and thus represents the greatest reservoir of energy in the body.

On a cellular level, adipocytes are spherical cells wherein the intercellular space is filled by a large vacuole filled with triglycerides. Adipocytes can change volume rapidly. In fact, these cells may reach, depending on the circumstances, 40 μm to 120 μm in diameter, which corresponds to a 27-fold increase in volume. In some extreme cases, this increase may be up to 40-fold. In this way, the adipocyte has the main energy role in the body as it is capable of storing (uptake or lipogenesis) or, conversely, mobilising (lipolysis) triglycerides, major sources of energy in the body, rapidly.

Lipogenesis involves the synthesis of triacylglycerols which is the result of the esterification of glycero-3-phosphate by activated fatty acids; conversely, lipolysis corresponds to the hydrolysis of the triacylglycerols stored, into glycerol and fatty acids. Different mechanisms have been demonstrated, which control lipolysis and lipogenesis involving for example receptors such alpha-2 and/or beta-1 and -2 receptors, adenosine A1, prostaglandin E2, Y2 type YY and neuropeptide NPY receptors and also sex hormones.

Two main types of slimming active substances are known: lypolytic substances (acting on excess fat elimination) and liporeducing substances (combating fat formation)

a) Lipolytic substances
Caffeine (found in a large number of plants: green tea, guarana beans): inhibits phosphodiesterase, thus ensuring an optimal intracellular cAMP level, stimulates β receptors and inhibits lipoprotein lipase;
Rhodysterol (red seaweed extract): activates a receptors and promote caffeine penetration;
Palmitoyl-carnitine: accelerates combustion of fatty acids, improving the uptake thereof by mitochondria;
Alpha and gamma bioactive substances (obtained from a marine bacterium and a fungus, respectively) block α2 and NPY receptors;
Escin and gingko biloba: α2 blockers;
Sphingosine: limits glucose penetration

b) Liporeducing substances
Andiroba (triterpenes) and Carcina Cambogia: block conversion of pre-adipocytes into adipocytes
Rutin: (Ruta graveolens extract): isolates glucose and prevents the combination thereof with free fatty acids.

To these specific active substances, it is possible to add anti-infiltration and venotonic active substances, which are frequently combined with slimming active substances.

Anti-infiltration substances:
Viburnum (draining, decongestant, activates caffeine, anti-radical, firming effect)
Ivy (anti-inflammatory, anti-swelling, analgesic)
Arnica (anti-swelling, soothing)
Pisola (anti-radical, accelerates drainage)
Wild pansy (important role in hydration balance)
Fucus vesiculosis (anti-swelling, soothing)

Venotonic substances:
Ruscus (vitamin P action, vascular stimulant)
Gingko biloba (combats vascular and capillary stasis)
Escin (improvement in venous tonicity, modifies capillary permeability)

Finally, formulations comprising these known slimming active substances may be completed by remodelling and smoothing active substances making it possible to combat slackening of the skin.

The slimming active substances according to the prior art are generally administered topically.

Food supplements comprising a combination a vitamins, trace elements and essential fatty acids are already known in the prior art.

For example, the international application Wo 91/11117 describes a formulation, intended to be administered orally, containing vitamins (B1, B2, B3, B5, B6, B8, B9, B12, C, D3, K1) and minerals (boron, calcium, chromium, copper, iodine, iron, magnesium, manganese, molybdenum, potassium, selenium, silica, vanadium and zinc) in combination with antioxidants and fish oils. This oral formulation is intended to prevent cancer and cardiovascular disease.

U.S. Pat. No. 6,071,544 describes the use of Q-3 and Q-6 fatty acids, to promote weight loss in cats, in an oral formulation, comprising in combination vitamins (A, B1, B2, B3, B5, B6, B7, B8, B9, B12, C, D3 and E) and minerals (calcium, cobalt, copper, iron, magnesium, manganese, phosphorus, potassium, selenium and zinc). No effect on slackening of skin is observed.

Although knowledge of adipocytic lipolysis and lipogenesis control mechanisms has improved very markedly, satisfactory slimming active substances are always required. In addition, it is not easy to determine a satisfactory combination of slimming active ingredients and remodelling and smoothing active ingredients. Therefore, at the present time, there is a genuine demand for the preparation of oral formulations, particularly food supplements, making it possible to promote slimming effectively and, at the same time, combat slackening of the skin.

The invention firstly relates to the use of a combination comprising:
a mixture of group B vitamins and derivatives thereof;
at least one trace element, in salt or complex form, chosen from the group consisting of zinc, magnesium, copper, selenium, chromium, iron, manganese and mixtures thereof;
at least one vegetable and/or animal oil comprising at least one essential fatty acid chosen from Ω-3 fatty acids and Ω-6 fatty acids;
and at least one excipient suitable for oral administration

for the manufacture of a food supplement, administered orally, for the treatment of excess weight localised in a surface area and simultaneously of slackening of the skin localised in the same area.

The invention also relates to a cosmetic treatment method of excess weight localised in a surface area and simultaneously of slackening of the skin localised in the same area, comprising the oral administration of a combination comprising:
a mixture of group B vitamins and derivatives thereof;
at least one trace element, in salt or complex form, chosen from the group consisting of zinc, magnesium, copper, selenium, chromium, iron, manganese and mixtures thereof;
at least one vegetable and/or animal oil comprising at least one essential fatty acid chosen from Ω-3 fatty acids and Ω-6 fatty acids;
and at least one excipient suitable for oral administration.

The invention finally relates to a food supplement, intended for oral administration, consisting of a combination consisting of:

a) a mixture of group B vitamins and derivatives thereof;

b) at least one trace element, in salt or complex form, chosen from the group consisting of zinc, magnesium, copper, selenium, chromium, iron, manganese and mixtures thereof;

c) at least one vegetable and/or animal oil comprising at least one essential fatty acid chosen from Ω-3 fatty acids and/or Ω-6 fatty acids;

d) if applicable, a plant enzyme advantageously chosen from the group comprising papain, bromelain and ficin;

e) and at least one excipient suitable for oral administration.

The vitamins found in the food supplement according to the invention are exclusively group B vitamins.

Within the scope of the present description, the mass ratios are determined, unless specified otherwise, on the basis of the weight by mass of the compounds (fatty acids, vitamins, trace elements) alone. In this way, for trace elements, the weight of the trace element alone is used as a basis and not the weight of the salt or complex thereof (for example, weight of zinc in zinc sulphate). Similarly, for vitamins, the weight of vitamin contained in the derivative is used as a basis (for example, weight of vitamin B3 contained in commercial nicotinamide). For Ω-3 and Ω-6 essential fatty acids, the content thereof in vegetable and/or animal oils is used as a basis.

The combination or food supplement advantageously comprises said vitamins, said trace elements and said essential fatty acids in a (Ω-3 and/or Ω-6 essential fatty acids):(group B vitamins+trace element(s)) mass ratio between 3 and 6, advantageously between 3.5 and 5. This mass ratio is preferentially approximately 4.

In the combination or in the food supplement, said Ω-3 and Ω-6 essential fatty acids are advantageously present in a Ω-3:Ω-6 mass ratio between 1 and 5, advantageously 1 and 4, more advantageously 1 and 3, even more advantageously between 1.4 and 2.1. This ratio is advantageously approximately 2.

The combination or the food supplement advantageously comprises said vitamins and said trace elements in a (group B vitamins):(trace elements) mass ratio between 0.2 and 1, advantageously between 0.4 and 0.6. This mass ratio is preferentially approximately 0.5.

The Ω-3 essential fatty acids present in the combination or in the food supplement are advantageously eicosapentaenoic acid (EPA) and/or docosahexaenoic acid (DHA). The combination or food supplement advantageously comprises more EPA than DHA, more advantageously at least 2 times more, preferentially at least 3 times more, more preferentially at least 4 times more, or at least 5 times more EPA than DHA.

The Ω-6 essential fatty acids present in the combination or in the food supplement are advantageously linoleic acid and/or gamma-linoleic acid (GLA). The combination or food supplement advantageously comprises more linoleic acid than gamma-linoleic acid, more advantageously the linoleic acid:gamma-linolenic acid mass ratio is between 1.2 and 2, more advantageously between 1.6 and 1.9.

The group B vitamins are advantageously chosen from the group consisting of vitamins B2, B3, B5, B6, B8, B9, B12 and mixtures thereof. The sources of these vitamins are advantageously:
vitamin B2: riboflavin, riboflavin-5′-sodium phosphate
vitamin B3 (niacin): nicotinic acid, nicotinamide
vitamin B5 (pantothenic acid): calcium D-pantothenate, sodium D-pantothenate, dexpantothenol
vitamin B6: pyroxidine hydrochloride, pyroxidine-5′-phosphate
vitamin B8 (biotin): D-biotin
vitamin B9 (folic acid): pteroylmonoglutamic acid, folic acid
vitamin B12 (cobalamins): cyancobalamin, hydroxyocobalamin

The group B vitamins and derivatives thereof are advantageously chosen from the group consisting of riboflavin, nicotinamide, calcium pantothenate, pyridoxine hydrochloride, biotin, folic acid, cobalamin, and mixtures thereof.

The zinc advantageously comes in the form of zinc salt or in the form of a complex, in particular in the form of zinc acetate, zinc chloride, zinc citrate, zinc gluconate, zinc lactate, zinc oxide, zinc carbonate or zinc sulphate. In an advantageously alternative embodiment of the invention, the zinc is introduced in the form of zinc sulphate. In this case, the food supplement according to the invention advantageously comprises 15 to 25 mg, per dose of zinc sulphate.

Magnesium is used to convert the group B vitamins into active form. It is advantageously introduced into the combination or the food supplement in the form of magnesium acetate, magnesium carbonate, magnesium chloride, citric acid magnesium salts, magnesium gluconate, magnesium glycerophosphate, orthophosphoric acid magnesium salts, magnesium lactate, magnesium hydroxide, magnesium oxide or magnesium sulphate, the magnesium is preferentially introduced in the form of magnesium oxide. In this case, the food supplement according to the invention advantageously comprises 30 to 35 mg, per dose of magnesium oxide.

Copper is advantageously introduced into the combination or food supplement in the form of copper carbonate, copper citrate, copper gluconate, copper sulphate or copper-lysine complex.

Selenium is advantageously introduced into the combination or food supplement in the form of yeast extracts nourished with selenium, sodium selenate, sodium hydrogen selenite or sodium selenite.

Chromium is advantageously introduced into the combination or food supplement in the form of chromium (III) chloride or chromium (III) sulphate.

Iron is advantageously introduced into the combination or food supplement in the form of iron carbonate, iron citrate, ammonium iron citrate, iron gluconate, iron fumarate, sodium iron diphosphate, iron lactate, iron sulphate, iron diphosphate (iron pyrophosphate) or iron saccharate.

Manganese is advantageously introduced into the combination in the form of manganese carbonate, manganese chloride, manganese gluconate, manganese glycerophosphate or manganese sulphate.

The combination or food supplement preferentially comprises at least zinc and magnesium, as trace elements; according to an advantageous alternative embodiment of the invention, the only trace elements present in the combination of food supplement are zinc and magnesium.

The oils used in the combination or food supplement are oils rich in omega-3 (walnut, rapeseed, wheat germ, soy, canola, linseed, fish, krill oils) and/or omega-6 (grape seed, blackcurrant, sunflower, walnut, corn, borage, evening primrose, safflower, wheat gum, soy, sesame, rapeseed, peanut, hazelnut, olive oils). The oils advantageously used within the scope of the invention are borage and/or fish oil.

According to an advantageous alternative embodiment of the invention, the combination or food supplement also comprises a plant enzyme, particularly a plant enzyme chosen from the group consisting of papain, bromelain and ficin.

The food supplement according to the invention advantageously consists of a 365 mg mixture consisting of:

a) 15 to 30 mg of a mixture of group B vitamins and mixtures thereof chosen from the group consisting of riboflavin, nicotinamide, calcium pantothenate, pyridoxine hydrochloride, biotin, folic acid and cobalamins;

b) 38 to 61 mg of a mixture of magnesium oxide and zinc sulphate;

c) 200 to 245 mg of a mixture of borage oil and fish oil;

d) 20 to 30 mg of a pineapple extract concentrated with bromelain, and

e) q.s. excipient(s) suitable for oral administration.

The excipients that can conventionally be used are glyceryl monostearate, rapeseed or soy lecithin, vegetables oils (soy, sunflower), hydrogenated or partially hydrogenated vegetables oils, waxes such as beeswax.

The cosmetic treatment method according to the invention may prove to be particularly advantageous for pregnant women or women having given birth less than 6 months previously, who may be breastfeeding. In this way, the present invention also relates to a cosmetic treatment method to combat excess weight localised in a surface area and simultaneously to combat slackening of the skin localised in the same area in pregnant women or women having given birth less than 6 months previously (who may be breastfeeding). In fact, one of the advantages of the combination according to the invention is the absence of active ingredients contraindicated for women having given birth, particularly for breastfeeding women. In addition, the combination or food supplement makes it possible to supply women having recently given birth with group B vitamins, breastfeeding or pregnant women being considered as an at-risk group for group B vitamin deficiency, particularly vitamins B2, B5, B6 and B9.

The term “slimming” or “combating localised excess weight” according to the present invention refers to an action making it possible to prevent or at the very least reduce subcutaneous fat formation as described above. In this way, fat storage (lipogenesis) is limited significantly and/or fat release (lipolysis) is stimulated significantly. This action promotes a reduction in adipose tissue and is particularly conveyed by a reduction in unsightly excess weight or reserves, by a slimming of the FIGURE, by an acceleration in surplus elimination, by an improved definition of the outline of the body or a remodeled FIGURE.

The term “combating localised slackening of the skin” according to the invention refers to an action making it possible to:
reinforce the epidermal barrier and remodel the epidermis, particularly by activating the keratinocyte metabolism (proliferation/differentiation); and/or
redensify the skin (the skin's elasticity is restored), particularly by activating the fibroblast metabolism (proliferation/total protein synthesis) and/or by stimulating the synthesis of key extracellular matrix molecules (collagen I, elastin, fibronectin); and/or
reinforce the dermoepidermal junction, particularly by stimulating collagen VII synthesis.

The term “cosmetic treatment method to combat localised excess weight and localised slackening of the skin”, according to the present invention, refers to the application of a cosmetic treatment making it possible to measure the action described above visibly.

In this way, the combination or food supplement according to the invention may be used to treat areas of the skin liable to form said localised excess weight, i.e. areas where said excess weight has already formed or is in the process of forming.

The various compounds in the combination may be administered simultaneously (in the same capsule for example) or as combination products for a separate use or a use spread over time. The various compounds of the combination are advantageously administered simultaneously (in the same capsule for example).

The food supplement or combination is formulated to be administered orally. In this way, it may be presented in the form of a capsule, soft capsule, tablet, granule, advantageously in the form of a capsule. If the food supplement or combination is presented in the form of a soft capsule, the coating of said soft capsules may particularly contain animal gelatine such as fish gelatine, glycerine, or a material or plant origin such as a cellulose or starch derivative, or a plant protein. If the food supplement or combination is presented in the form of a soft capsule, tablet, or granule, the mixture of active substances may be fixed on a powder substrate such as silica, cellulose, and maltodextrin.

The recommendations for use are 2 doses per day.

The food supplement according to the present invention may advantageously comprise any suitable vehicle or excipient, acceptable from a nutriceutical point of view, along with conventional additives, known to those skilled in the art.

The food supplement and the combination may be used in combination with other slimming-action active substances such as lipolytic and liporeducing substances simultaneously, separately or spread over time.

The lipolytic type slimming active substance may be chosen from: caffeine, rhodysterol, palmitoyl-carnitine, alpha and gamma bioactive substances, escin, gingko biloba and sphingosine. The liporeducing type slimming active substance may be chosen from: andiroba, Garcinia Cambogia, rutin.

It is also possible to use simultaneously, separately or spread over time, one or two anti-infiltration or venotonic active substances in addition to the combination or food supplement according to the invention. The anti-infiltration or venotonic active substances may be chosen from: viburnum, ivy, arnica, pisola, wild pansy, Fucus vesicolosus, ruscus, gingko biloba and escin.

The examples hereinafter illustrate the invention.

### EXAMPLE 1

### Food Supplement

The food supplement according to example 1 complies with the following composition by weight:

TABLE 1
food supplement composition

| | | Equivalent |
|---|---|---|
| | | weight/vitamin or |
| | | trace element |
| Compounds | Weight (mg) | alone |
| Borage oil | 80-105 | |
| containing 20% | | |
| GLA | | |
| Fish oil | 100.000-140.000 | |
| Magnesium oxide | 24.900-33.200 | i.e. 15-20 mg |
| | | magnesium |
| Pineapple extract | 20.000-30.000 | |
| concentrated with | | |
| bromelain | | |
| Riboflavin | 0.600-1.000 | i.e. 0.5-0.9 mg |
| | | of vitamin B2 |
| Nicotinamide | 5.500-11.000 | i.e. 5-10 mg of |
| | | vitamin B3 |
| Calcium | 1.419-4.966 | i.e. 1-3.5 mg of |
| pantothenate | | vitamin B5 |
| Pyroxidine | 0.790-1.580 | i.e. 0.5-1 mg of |
| hydrochloride | | vitamin B6 |
| Biotin | 6.500-10.400 | i.e. 50-80 μg of |
| | | vitamin B8 |
| Folic acid | 0.098-0.141 | i.e. 70-100 μg of |
| | | vitamin B9 |
| Vitamin B12 | 0.103-0.650 | i.e. 0.1-0.5 μg |
| | | of vitamin B12 |
| Zinc sulphate | 13.75-27.49 | i.e. 5-10 mg of |
| | | zinc |
| Excipients: | q.s. 365.000 mg | |
| glyceryl | | |
| monostearate, | | |
| rapeseed lecithin | | |

The food supplement is in the form of a soft capsule. For this specific soft capsule, the recommended dose is two capsules per day, preferentially one in the morning and one in the evening.

### EXAMPLE 2

### In Vitro Study

The purpose of this test is to demonstrate the effect of the combination as described in example 1 on type I collagen synthesis in a fibroblast model in culture.

Materials and methods: Normal human dermal fibroblasts were inoculated, at the passage P10, in 96-well plates, in DMEM (Dulbeco Modified Eagle Medium) medium with GlutaMAX™ 1,1000 mg/l D-Glucose, Sodium Pyruvate, supplemented with 10% Foetal Calf Serum+antibiotics.

After 24 hours of incubation at 37° C., 5% CO₂ in a humidified atmosphere, the cells were rinsed twice with PBS and incubated for 48 hours with or without the combination according to example 1.

At the end of the treatment, the total intracellular protein concentration is determined using the Lowry method and, in parallel, the type I collagen released by the cells is assayed in the culture supernatants by means of an ELISA technique.

For each treatment condition, the type I collagen concentration is referred to the quantity of total proteins, and the results are expressed in ng of type I collagen per μg of proteins.

The increase in the release of type I collagen induced by the treatment with the combination according to example 1 with respect to the control (untreated) cells is calculated as follows:

$$\% increase = 100 \times \frac{Treatment collagen I - Control collagen I}{Control collagen I}$$

The significance of the results is analysed by means of a Student's t test.

Four tests were performed. The mean of the tests and the standard deviation are given in table 2 below:

TABLE 2

Collagen I (ng/μg protein)

| | | Standard | % | |
|---|---|---|---|---|
| | Mean | deviation | increase | Student |
| Control cells | 6.862 | 1.073 | 0 | |
| Combination | 39.836 | 1.475 | 481 | 0.000 |
| according to | | | | |
| example 1 | | | | |

Conclusion:

The combination according to example 1 makes it possible, under our experimental conditions, to increase type I collagen synthesis by dermal fibroblasts in culture by 481% (p<0.001).

### EXAMPLE 3

### Clinical Study

The purpose of the test will be to evaluate the benefit of supplementation with the food supplement according to example 1 in post-childbirth body remodelling. It will be analysed on postpartum women by comparing a treated group to an untreated control group. This test is performed by dermatologists or midwives.

Treatment: intake or not of one capsule in the mornings and evenings of the food supplement as described in example 1 for 3 months.

Subjects: women, breastfeeding or not, having given birth at least 2 months and not more than 3 months previously. These women did not follow in parallel any other slimming treatment and/or treatment intended to remodel the skin. In addition, they did not follow such a treatment after giving birth and before the test was conducted.

Tables 3 and 4 below report the variations (in %) of the waist (table 3) and thigh (table 4) circumference measurements for the treated and untreated groups.

TABLE 3
variation in waist circumference

| | difference in percentage: waist | | | |
|---|---|---|---|---|
| | circumference measured after X days | | | |
| | of treatment - initial waist circumference | | | |
| | 30 | 45 | 60 | 90 |
| | days | days | days | days |
| Untreated group | −1.1% | +1.5% | −0.9% | −4.9% |
| Treated group | −2.4% | −3.1% | −3.8% | −5.7% |

TABLE 4
variation in thigh circumference

| | difference in percentage: thigh | | | |
|---|---|---|---|---|
| | circumference measured after X days | | | |
| | of treatment - initial thigh circumference | | | |
| | 30 | 45 | 60 | 90 |
| | days | days | days | days |
| Untreated group | +3.4% | +0.6% | −1% | −2.2% |
| Treated group | −2.1% | −3.6% | −2.6% | −4.1% |

These parameters progress more favourably for the treated group than for the untreated group. These results demonstrate the efficacy of twice-daily administration of capsules according to example 1 on reducing waist and thigh circumference measurements.

Table 5 below summarises the results of the self-evaluations of the subjects. There is a statistically significant difference between the treated group and the untreated group, in favour of the treated group. In other words, these results demonstrate the efficacy of a twice-daily dose of food supplement according to example 1.

TABLE 5

| | Treated | Untreated |
|---|---|---|
| Your skin's elasticity has been | 73.7% | 33.3% |
| restored | | |
| Your skin's suppleness has been | 68.4% | 26.7% |
| restored | | |
| Your skin's tonicity has been | 73.7% | 33.3% |
| restored | | |
| Slackening of skin in the abdominal | 68.4% | 46.7% |
| area has decreased | | |
| Firming is visible in the buttock | 68.4% | 40.0% |
| area | | |
| Firming is visible in the thigh area | 57.9% | 40.0% |
| Your body tissue firmness has | 73.7% | 53.3% |
| improved | | |
| The skin's tautness has been | 63.2% | 46.7% |
| restored | | |
| Your skin is firmer | 68.4% | 53.3% |
| The skin's softness to the touch has | 73.7% | 40.0% |
| improved | | |
| The skin progressively appears | 68.4% | 40.0% |
| shinier | | |
| The quality of the skin is improved | 73.7% | 53.3% |
| The skin is remodelled | 78.9% | 46.7% |
| The skin is more moisturised | 84.2% | 53.3% |
| Dryness of the skin has been | 78.9% | 60.0% |
| absorbed | | |
| Your figure looks slimmer | 57.9% | 40.0% |
| Your figure looks firmer | 57.9% | 33.3% |
| Your curves have decreased | 57.9% | 40.0% |
| The size of pregnancy stretch marks | 76.5% | 35.7% |
| has decreased | | |

Twice-daily administration of a food supplement according to example 1:
makes it possible to reduce the waist circumference and thigh circumference significantly;
induces a significant improvement in skin elasticity, suppleness and tonicity and reduces slackening of the skin in parallel;
is correlated with overall firming of the skin combined with a slimmer FIGURE and reduced curves;
makes it possible to decrease the size of pregnancy stretch marks;
makes it possible to improve the appearance of the skin.

1. A method for the treatment of excess weight localised in a surface area and simultaneously of slackening of the skin localised in the same area comprising the oral administration to a person in need thereof of a combination comprising:
a mixture of group B vitamins and derivatives thereof;
at least one trace element, in salt or complex form, chosen from the group consisting of zinc, magnesium, copper, selenium, chromium, iron, manganese and mixtures thereof;
at least one vegetable and/or animal oil comprising at least one essential fatty acid chosen from Ω-3 fatty acids and Ω-6 fatty acids;
and at least one excipient suitable for oral administration.

2. The method of claim 1, wherein the combination comprises said vitamins, said trace elements and said essential fatty acids in a (Ω-3 and/or Ω-6 essential fatty acids):(group B vitamins+trace element(s)) mass ratio between 3 and 6.

3. The method of claim 1, wherein the combination comprises said Ω-3 and Ω-6 essential fatty acids in a Ω-3:Ω-6 mass ratio between 1 and 5.

4. The method of claim 1, wherein the combination comprises said vitamins and said trace elements in a (group B vitamins):(trace elements) mass ratio between 0.2 and 1.

5. The method of claim 1, wherein the group B vitamins and derivatives thereof are chosen from the group consisting of riboflavin, nicotinamide, calcium pantothenate, pyroxidine hydrochloride, biotin, folic acid, cobalamin, and mixtures thereof.

6. The method of claim 1, wherein the combination comprises, as trace elements, zinc and magnesium.

7. The method of claim 1, wherein the oil used in the combination is borage oil and/or fish oil.

8. The method of claim 1, wherein the combination further comprises a plant enzyme, particularly a plant enzyme chosen from the group consisting of papain, bromelain and ficin.

9. A food supplement, intended for oral administration, comprising:
a) a mixture of group B vitamins and derivatives thereof;
b) at least one trace element salt or complex, said trace element being chosen from the group consisting of zinc, magnesium, copper, selenium, chromium, iron, manganese and mixtures thereof;
c) at least one vegetable and/or animal oil comprising at least one essential fatty acid chosen from Ω-3 fatty acids and/or Ω-6 fatty acids;
d) if applicable, a plant enzyme chosen from the group comprising consisting of papain, bromelain and ficin;
e) and at least one excipient suitable for oral administration.

10. The food supplement according to claim 9, wherein said vitamins, said trace elements and said essential fatty acids are introduced in a (Ω-3 and/or Ω-6 essential fatty acids):(group B vitamins+trace element(s)) mass ratio between 3 and 6.

11. The food supplement according to, wherein the Ω-3 and Ω-6 essential fatty acids are introduced in a Ω-3:Ω-6 mass ratio between 1 and 5.

12. The food supplement according to claim 9, wherein vitamins and trace elements are introduced in a (group B vitamin):(trace elements) mass ratio between 0.2 and 1.

13. The food supplement according to claim 9, wherein the group B vitamins and derivatives thereof are chosen from the group consisting of riboflavin, nicotinamide, calcium pantothenate, pyroxidine hydrochloride, biotin, folic acid, cobalamin, and mixtures thereof.

14. The food supplement according to claim 9, wherein the combination comprises, as trace elements, zinc and magnesium.

15. The food supplement according to claim 9, wherein it consists essentially of a 365 mg mixture comprising:
a) 15 to 30 mg of a mixture of group B vitamins and mixtures thereof chosen from the group consisting of riboflavin, nicotinamide, calcium pantothenate, pyridoxine hydrochloride, biotin, folic acid and cobalamins;
b) 38 to 61 mg of a mixture of magnesium oxide and zinc sulphate;
c) 200 to 245 mg of a mixture of borage oil and fish oil;
d) 20 to 30 mg of a pineapple extract concentrated with bromelain, and
e) q.s. excipient(s) suitable for oral administration.

16. (canceled)

17. The method according to claim 1, wherein the combination consist essentially of a 365 mg mixture comprising:
a) 15 to 30 mg of a mixture of group B vitamins and mixtures thereof chosen from the group consisting of riboflavin, nicotinamide, calcium pantothenate, pyridoxine hydrochloride, biotin, folic acid and cobalamins;
b) 38 to 61 mg of a mixture of magnesium oxide and zinc sulphate;
c) 200 to 245 mg of a mixture of borage oil and fish oil;
d) 20 to 30 mg of a pineapple extract concentrated with bromelain, and
e) q.s. excipient(s) suitable for oral administration.
